(19) United States
(12) Patent Application Publication
(10) Pub. No.: US 2023/0088382 A1
(43) Pub. Date: Mar. 23, 2023

(54) Methods Circuits Systems & Functionally Associated Machine Executable Code to Perform Dynamic Scheduling & Management of Resources

(71) Applicants: Avner Yoffe, Tel Aviv (IL); Matan Croitoriu, Herzeliya (IL)

(72) Inventors: Avner Yoffe, Tel Aviv (IL); Matan Croitoriu, Herzeliya (IL)

(21) Appl. No.: 17/933,106

(22) Filed: Sep. 18, 2022

Related U.S. Application Data
(60) Provisional application No. 63/261,351, filed on Sep. 18, 2021.

(51) Int. Cl. G16H 40/20

(52) U.S. Cl. CPC G16H 40/20

(57) ABSTRACT

Disclosed are method, devices, systems and functionally associated machine executable code for dynamically and anonymously trading and scheduling appointments between parties over a computerized resource scheduling system.

### RELATED APPLICATIONS

The present U.S. Utility Application claims priority from U.S. Provisional Patent Application No. 63/261,351, filed on the 18th of Sep. 2021, the entirety of which application is hereby incorporated by reference in its entirety.

### FIELD OF THE INVENTION

The present application relates generally to the field of computerized resource scheduling systems. More specifically, the present application discloses and covers computerized scheduling systems for enabling AI driven scheduler, rescheduler and peer to peer (or client or patient) appointment/shifts swapping.

### BACKGROUND OF THE INVENTION

Computerized scheduling systems have been in use for at least 20 years. These systems record and, in some cases, notify participants of an appointment for some service to be provided or for some other type of interaction. Despite the great improvement in record keeping and these systems have provided, they are still quite static and fail to address a common phenomenon of “no-shows”, which phenomena contribute to a considerable waste of resources on the part of the service provider.

These days, medical services and caregivers' appointments are set on-line via phone calls or in-person. Although reminders for appointment are sometimes sent via an email or SMS, they are nevertheless “hard to change”, especially if the service or associated resource is greatly needed, very popular or of a rare/limited/singular nature, such the time of an expert physician whose schedule is most likely to be fully booked for a long periods in advance.

Due to inefficiencies present in today's scheduling systems, people who are in need of a particular limited resource, e.g. appointment, can't get one. While other people who were lucky enough to get an appointment scheduled for them long in advance may miss their appointment completely, leaving a valuable resource to go unutilized.

### SUMMARY OF THE INVENTION

Embodiments of the present invention include methods, device and systems and machine executable code for addressing and overcoming the above-mentioned drawbacks of existing computerized resource scheduling systems. More specifically, the present invention includes methods, circuits, systems & functionally associated machine executable code to perform dynamic scheduling and management of resource such as appointments and work shifts. The provided solution includes an innovative and flexible mechanism for scheduling, rescheduling and preparing for a medical appointment, either as doctor or a patient.

Embodiments of the present invention enable mechanisms for generating and exchanging offers between peers rescheduling a set of appointments, wherein the appointments are treated as a transferable resource. There is provided an appointment swapping system where the identities of the parties attempting to swap an appointment or agreeing to reschedule an appoint are anonymous, according to embodiments. As a consequence of the schedule swapping system, there may be a reduction in potential waste of the managed resource/appointment, also enabling a win-win-win scenario for the service provider and its users.

Embodiments of the present invention may disclose and cover computerized scheduling systems for enabling AI driven scheduler, re-scheduler and peer to peer (or client or patient) appointment/shifts swapping.

The medical appointment as a resource use case, described in this application, is only one embodiment of many for the innovations presented herein. Other use cases, appointment or not, of the presently described innovations can be applied to many kinds of scheduled resource/shift/application, such as:
1. Government and organization appointments.
2. Post-office, service providers, banks, garages, stores.
3. Meeting/appointment management services.
4. Entertainment and culinary industry—waitresses, kitchen workers, bartenders etc.
5. Governmental services shift workers—police, fire departments, army,
6. Medical services:
   a. Patients appointments swapping
   b. Caregivers (physicians, nurses, ambulance, etc.) shifts swapping
7. Shift workers scheduling and swapping.

Embodiments of the present invention may disclose and cover computerized scheduling systems for enabling AI driven scheduling, re-scheduling and peer to peer (or client or patient) appointment/shifts swapping. While conventional optimization methods, rely on:
1. Personal reminders sent to users, sometimes arousing feelings of guilt for no-shows.
2. Online calendar management platforms

The present invention may employ an AI matching engine to match parties for a swapping transaction. Additionally, according to embodiments, the AI may be employed to prioritize possible resource receivers based on various parameters likelihood of timely appearance, likelihood or relevance of the appointment, and many other factors associated with a person's actual needs. According to further embodiments, the AI may scan through a database of possible transactions partners and may structure a proposed multi-party swapping transaction between two or more parties concurrently.

Embodiments of the present invention may include innovative system modifications to the existing state of the art in scheduling and appointments/shifts management systems, as partially listed below.

1. Swapping and bidding mechanism:
When two or more peers are scheduled to the same specialist/service, And at least one of them wants to reschedule his appointment, He can either bid or offer his already allocated appointment's time-slot to his peers anonymously, without any additional human intervention by the scheduled resource manager (e.g. receptionist, medical secretary), and get a notification when a potential swap is available, and suits his preset preferences/constraints.

2. Proactive engagement:
The system can either actively initiate interaction with the user (through the available communication channels/methods) and ask for the user's status regarding appointment arrival or follow a user rescheduling request initiated by the user.

3. Collection and analysis of historical user's actions, and management of “credit scores”:
Based on a user's historical behaviour, the system can predict one's preferred time slots, probability of arrival and so on.

4. Severity of condition (mostly for the medical sector):
Based on the patient's health condition, the system can predict the likelihood of a no-show.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a functional block diagram with data flow indicators of an exemplary computerized rescheduling and appoints swapping system in accordance of embodiment of the present invention;

FIG. 2A show an exemplary data structure including appointment data prior to a processing in accordance with embodiments of the present invention; and

FIG. 2B show an exemplary data structure including appointment data during swap processing in accordance with embodiments of the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION

In the following detailed description, numerous specific details are set forth in order to provide a thorough understanding of the invention. However, it will be understood by those skilled in the art that the present invention may be practiced without these specific details. In other instances, well-known methods, procedures, components and circuits have not been described in detail so as not to obscure the present invention.

Unless specifically stated otherwise, as apparent from the following discussions, it is appreciated that throughout the specification discussions utilizing terms such as “processing”, “computing”, “calculating”, “determining”, or the like, may refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

In addition, throughout the specification discussions utilizing terms such as “storing”, “hosting”, “caching”, “saving”, or the like, may refer to the action and/or processes of ‘writing’ and ‘keeping’ digital information on a computer or computing system, or similar electronic computing device, and may be interchangeably used. The term “plurality” may be used throughout the specification to describe two or more components, devices, elements, parameters and the like.

Some embodiments of the invention, for example, may take the form of an entirely hardware embodiment, an entirely software embodiment, or an embodiment including both hardware and software elements. Some embodiments may be implemented in software, which includes but is not limited to firmware, resident software, microcode, or the like. Furthermore, some embodiments of the invention may take the form of a computer program product accessible from a computer-usable or computer-readable medium providing program code for use by or in connection with a computer or any instruction execution system. For example, a computer-usable or computer-readable medium may be or may include any apparatus that can contain, store, communicate, propagate, or transport the program for use by or in connection with the instruction execution system, apparatus, or device.

In some embodiments, the medium may be an electronic, magnetic, optical, electromagnetic, infrared, or semiconductor system (or apparatus or device) or a propagation medium. Some demonstrative examples of a computer-readable medium may include a semiconductor or solid-state memory, magnetic tape, a removable computer diskette, a random-access memory (RAM), a read-only memory (ROM), any composition and/or architecture of semiconductor based Non-Volatile Memory (NVM), any composition and/or architecture of biologically based Non-Volatile Memory (NVM), a rigid magnetic disk, and an optical disk. Some demonstrative examples of optical disks include compact disk—read only memory (CD-ROM), compact disk—read/write (CD-R/W), and DVD.

In some embodiments, a data processing system suitable for storing and/or executing program code may include at least one processor coupled directly or indirectly to memory elements, for example, through a system bus. The memory elements may include, for example, local memory employed during actual execution of the program code, bulk storage, and cache memories which may provide temporary storage of at least some program code in order to reduce the number of times code must be retrieved from bulk storage during execution.

In some embodiments, input/output or I/O devices (including but not limited to keyboards, displays, pointing devices, etc.) may be coupled to the system either directly or through intervening I/O controllers. In some embodiments, network adapters may be coupled to the system to enable the data processing system to become coupled to other data processing systems or remote printers or storage devices, for example, through intervening private or public networks. In some embodiments, modems, cable modems and Ethernet cards are demonstrative examples of types of network adapters. Other functionally suitable components may be used.

### Embodiments of the Present Invention May be Include and be Characterized by the Following Features

1. A software system which reduces appointments “no-shows” by enabling unprecedented ability to swap appointment's time slots between peers/service users.
2. A computerized scheduling system which enables its users to swap between their appointments anonymously a. Based on a software-based system which interfaces a new or existing scheduling system of a service provider
   3. AI driven scheduling mechanism based upon a machine learning and data rule sets which can aggregate data from the service provider backend, other hard data sources and the system itself
4. A bidding mechanism which can be used as additional service
a. This is useful when a potential time slot offered to be swapped, has more than single “swap approval”
b. Thus, offers more degrees of freedom to the bidder and the resource administrator.
5. An anonymization layer embedded in the implementation to meet privacy-related regulatory and legal requirements.
6. Either interface with an existing scheduling system or replace it completely
   a. The user can choose which layers can/should be implemented to suit his system requirements.
   b. For instance car garage owners can wave the need for dire privacy requirements, and use a “lean” module.
7. The system can offer an external front end utilizing SMS, emails, phone calls, social media, web, mobile & web messaging platforms (e.g. Whatsapp, Signal, Telegram) or additional communication channels, notify and update the users.

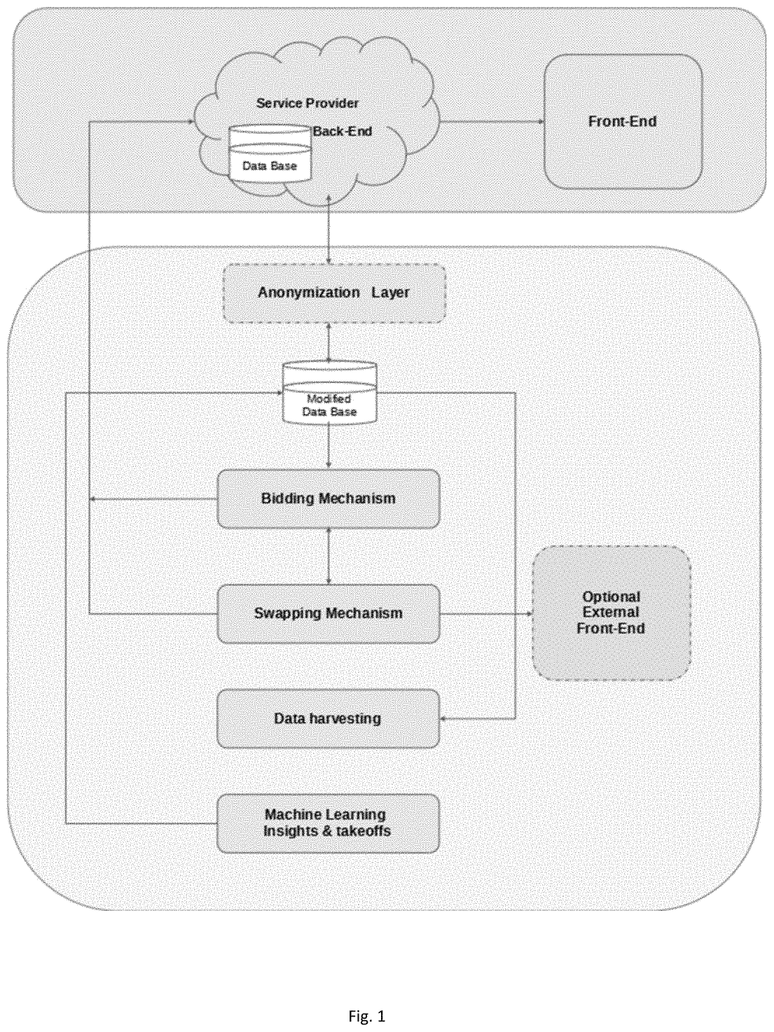

Turning to FIG. 1, there is shown a functional block diagram with data flow indication of an exemplary system in accordance with embodiments of the present invention. The exemplary system can be implemented off or on premise and can be implemented with the illustrated functional blocks, also referred to as modules, separately or combined. The modules are:
1. Service provider Backend interface
   a. Access the service provider database and schedules
   b. Arranging the relevant data for the optimization system
2. Anonymization layer (Optional)
   a. Gets the arranged data
   b. Clean any private data of users by mathematical/algorithm manipulation
3. Bidding Mechanism (Optional)
   a. Gets initial user preferences for an alternative appointment
   b. Seek potential “swappers”
   c. Sends notifications (by Frontend)
   d. Wait for approvals
   e. Notifies swapping layer
4. Swapping Mechanism layer
   a. Performs the swap in the database according to user priority and preference.
5. Data harvesting layer
   a. Responsible for collecting, sorting and indexing key parameters for real-time or future analysis.
6. Machine Learning and Behavioural Profiling layer
   a. Processes previously collected data by the “Data Harvesting Layer”, and transforms data into insights, using machine learning/or target specific optimized algorithms.
   b. Continuously provides insights and actionable takeoffs to the Service Provider.
   c. Optionally manages peers profiles by continuously tracking their behaviour and interactions with the system, which helps optimizing future “first schedule success/yield”.

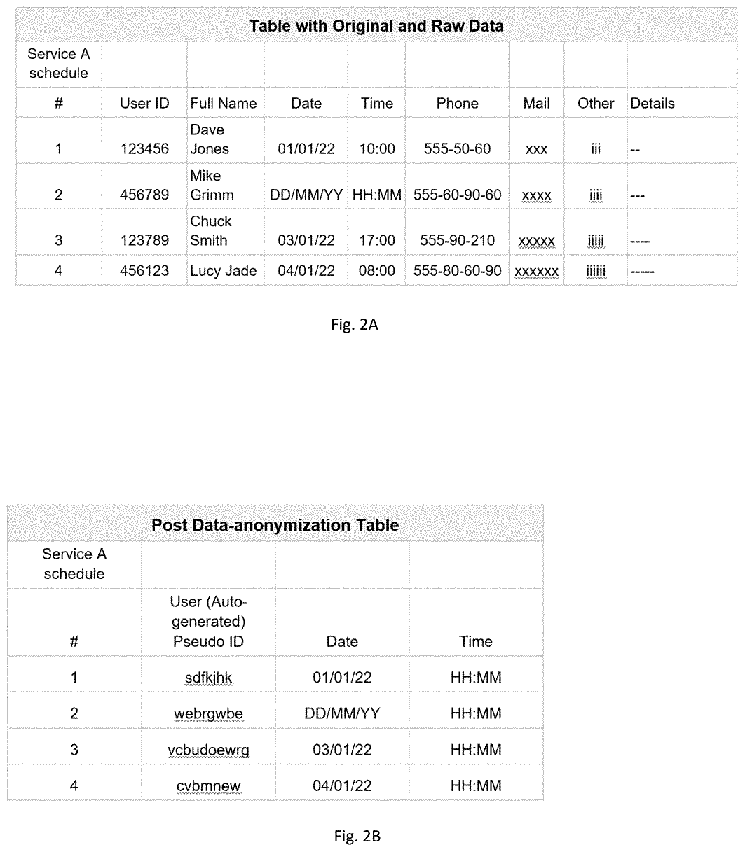

Turning now to FIG. 2A, there is shown an exemplary data structure of an appointment holder's data as it relates to the appointment in an open format.

Turning to FIG. 2B, there is shown exemplary appointment data after passing through the system.

After passing through the anonymization layer, a new hashed table comprised of pseudo data is generated and is used for swapping and bidding mechanisms. The following an exemplary process flow of an exemplary system in accordance with embodiments of the present invention:

Let us take Dave (#1) as a “dynamic rescheduling service subscriber” who wants to change his January 1st, 10:00 am appointment, and assume that using the conventional system, his earliest available appointment is March 7th.

Let us assume Chuck and Lucy are also subscribers.

Once Dave offers his appointment for potential swapping,

Chuck and Lucy —if enabled the option, will be notified with a potential sooner appointment is available (which is originally allocated for Dave).

If it suits their preference they can offer their appointment for a bid.

Dave, who was the process initiator will be notified of his potential available appointments, and can choose his preferred appointment.

This mechanism can be modified and implemented in various ways either automatic or manual.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

In the above description, numerous specific details were set forth in order to provide a thorough understanding of the invention. However, it will be understood by those skilled in the art that the present invention may be practiced without these specific details. In other instances, well-known methods, procedures, components and circuits have not been described in detail so as not to obscure the present invention.

The processes and displays presented herein are not inherently related to any particular computer, device, system or other apparatus. Various general-purpose systems may be used with programs in accordance with the teachings herein, or it may prove convenient to construct a more specialized apparatus to perform the desired method. The desired structure for a variety of these systems will appear from the description below. In addition, embodiments of the present invention are not described with reference to any particular programming language. It will be appreciated that a variety of programming languages may be used to implement the teachings of the inventions as described herein.

Functions, operations, components and/or features described herein with reference to one or more embodiments, may be combined or otherwise utilized with one or more other functions, operations, components and/or features described herein with reference to one or more other embodiments, or vice versa. While certain features of the invention have been illustrated and described herein, many modifications, substitutions, changes, and equivalents will now occur to those skilled in the art. It is, therefore, to be understood that the appended claims are intended to cover all such modifications and changes as fall within the true spirit of the invention.

1. A system for transferring a scheduled resource, said system comprising:
a scheduling database functionally associated with a web-application server through which access to a given resource can scheduled; and
a computerized swapping mechanism functionally associated with an anonymizer to, responsive to a request from a holder of a given appointment to access the given resources, reassigns the given appointment to another person who has indicated an interest in the given appoint.

2. The system according to claim 1, wherein the swapping mechanism includes a bidding function.

3. The system according to claim 1, wherein the swapping mechanism utilizes a machine learning module to reassign the given appointment.
